(19) United States
(12) Patent Application Publication
(10) Pub. No.: US 2020/0384160 A1
(43) Pub. Date: Dec. 10, 2020

(54) IMPROVED MAGNESIUM ALLOY AND PROCESS FOR MAKING THE SAME

(71) Applicant: Thixomat, Inc., Livonia, MI (US)

(72) Inventors: Raymond Decker, Ann Arbor, MI (US); Jacob Edick, North Muskegon, MI (US); Daniel LaCroix, Ypsilanti, MI (US); Stephen LeBeau, Northville, MI (US)

(21) Appl. No.: 16/971,579

(22) PCT Filed: Feb. 19, 2019
(86) PCT No.: PCT/US2019/018545

§ 371 Date: Aug. 20, 2020

Related U.S. Application Data
(60) Provisional application No. 62/632,600, filed on Feb. 20, 2018.

(51) Int. Cl. A61L 31/02; A61L 31/16; C22C 23/04

(52) U.S. Cl. CPC A61L 31/022; A61L 2400/12; C22C 23/04; A61L 31/16

(57) ABSTRACT

A strengthened Mg based alloy comprising Mg as a base element and at least two microalloying elements. A microstructure having at least one of dislocations, stacking faults, coherency strains, grain boundaries and dislocation domains decorated by segregation of microalloying elements. One of the microalloying elements is a large atom element having an atomic size larger than the atomic size of a Mg atom and another of the microalloying elements is a small atom element having an atomic size smaller than the atomic size of the Mg atom. The microstructure includes an absence of continuous films of grain boundary intermetallic compounds.

### CROSS REFERENCE TO RELATED APPLICATION

This application claim priority to U.S. provisional patent application No. 62/632,600, filed Feb. 20, 2018, the entire contents of which are herein incorporated by reference.

### BACKGROUND

Mechanical properties double those of bioabsorbable polymers (which typically have a tensile strength of less than 110 MPa) are required for structural bone fixation, both for surgical insertion and also to insure rigid fixation of the repaired bone array. Requisite strength levels are seen in magnesium (Mg) alloys that are hardened by aluminum (Al), which have strength levels greater than 200 MPa. However, Al is a suspected contributor to dementia, Alzheimer's disease and bone dissolution. Accordingly, an alternate strengthening mechanism is required for bioabsorbable Mg alloys.

### SUMMARY

In satisfying the above need, as well as overcoming the enumerated drawbacks and other limitations of the related art, the present invention provides a strengthened Mg based alloy comprising Mg as a base element and at least two microalloying elements. The microstructure of the Mg based alloy has at least one of dislocations, stacking faults, coherency strains, grain boundaries and dislocation domains decorated (DDD) by segregation of microalloying elements. Additionally, one of the microalloying elements is a large atom element having an atomic size larger than the atomic size of a Mg atom and another of the microalloying elements is a small atom element having an atomic size smaller than the atomic size of the Mg atom.

In another aspect, the Mg based alloy includes an additional microalloying element having separate nanometer-sized 3^{rd} phase particles that inhibit grain growth.

In a further aspect, the additional microalloying element is Mn and the separate nanometer-sized 3^{rd} phase particles are alpha Mn particles.

In an additional aspect, the 3^{rd} phase particles are in the range of 10 to 200 nanometers.

In another aspect, the 3^{rd} phase particles have a solvus at a higher temperature than equilibrium intermetallic compounds of the microstructure.

In a further aspect, the microstructure includes an absence of continuous films of grain boundary intermetallic compounds.

In an additional aspect, the microstructure includes an absence of denuded grain boundaries.

In yet another aspect, atoms of the large atom element have atomic radii of 173 Angstroms or more and atoms of the small atom element have atomic radii of 145 Angstroms or less.

In still a further aspect, the atoms of the large atom element have an electronegativity of 1.1 or less and atoms of the small atoms element have an electronegativity of 1.4 or more.

In an additional aspect, the large atom element is Ca and the small atom element is at least one of Zn and Mn.

In another aspect, the microalloying elements essentially consist of Zn, Ca and Mn in the ranges (weight %) of 0.7 to 1.8 Zn, 0.2 to 0.7 Ca and 0.2 to 0.7 Mn.

In yet a further aspect, the microalloying elements essentially consist of Zn, Ca and Mn in the ranges (weight %) of 0.5 to 2.0 Zn, 0.2 to 1.0 Ca and 0.2 to 1.0 Mn.

In an additional aspect, the base and microalloying elements are human body nutrients and are osteoconductive

In still another aspect, the Mg alloy is provided in the form of a bioabsorbable, animal, particularly human, body implant.

In a further aspect, the Mg alloy is provided in the form of a structural reinforcement device.

In yet an additional aspect, Mg allow retains dislocation and partial dislocation content of greater than 10¹³/m³.

In another aspect, the Mg alloy has a yield strength of greater than 220 MPa.

In a further aspect, the Mg alloy includes texture below a multiple of a random distribution (MRD) value of 5 as measured by electron diffraction and formability enhanced by an r value of less than 2 (as measured by width and thickness of deformed tensile samples).

In an additional aspect, strength of the Mg alloy is supplemented by intragranular GP zones (which are ordered arrays of big and small atoms on the basal plane of the Mg matrix, see FIGS. 2A, 2B and 3) of less than 100 nm in size and/or intragranular intermetallic particles (e.g, Ca₃Mg₆Zn₂) of less than 200 nm in size.

In a further aspect, strength of the Mg alloy is supplemented by coherency stresses arising from segregation of alloying elements. The high alloy volumes differ in lattice constants from the low alloy volumes, thus generating stresses when these volumes retain lattice coherency.

In another aspect of the invention, a method of processing a bioerodible magnesium alloy containing at least 95 weight percent magnesium in combination with microalloying elements for forming an endoprosthesis device is provided and includes the steps of forming one of an ingot or billet comprised of the magnesium alloy; strengthening the magnesium alloy by applying a deformation and segregation treatment, the treatment forming at one of dislocations, grain boundaries, stacking faults, clusters, GP zones and/or dislocation domains that are decorated by segregation of microalloying elements; at least one element of the microalloying elements is a large atom microalloying element having atoms with an atomic size larger than a magnesium atom; at least another element of the microalloying elements is a small atom microalloying element having atoms with an atomic size smaller than the magnesium atom, and forming in the microstructure separate grain growth inhibiting nanometer-sized 3^{rd} phase particles of an additional microalloying element.

In another aspect, one of a rod, wire, hollow tube and sheet is formed from the ingot or billet.

In a further aspect, the bioerodible magnesium alloy is formed into an endoprosthesis implant in the form of at one of a screw, plate, wire, mesh, scaffold and/or stent.

In an additional aspect, the large atom microalloying element is one or more of Ca, Sr, Ba, Na, K, RE and Y and the small atom microalloying element is of one or more of Zn, Mn, Sn, V, Cr, P, B, Si, Ag and Al.

In still another aspect, deformation is by at least one of cold drawing, cold stamping, cold stretching, cold swaging, cold spinning or cold rolling.

In yet a further aspect, deformation is by at least one of hot extrusion, hot rolling, hot pressing, hot swaging, hot spinning or hot forging wherein the rolls or dies are heated to 150 to 400° C. and the deformation is greater than 30%.

In an additional aspect, the forming step includes forming decorated dislocations of grain sizes less than 5 μm and decorated dislocation domains of less than 50 nanometers (see FIGS. 6 and 7).

In yet another aspect, the product of percent deformation×time (minutes)×temperature (° K) in the segregation treatment is between 5×10⁴ and 5.6×10⁵ for hot deformation and between 8×10⁴ and 21×10⁵ for cold deformation.

In still a further aspect, the deformation and segregation treatment includes cold working producing adiabatic heating resulting in segregation.

In an additional aspect, the additional microalloying element is Mn and the process of deformation and segregation treatment in the presence of the nanometer-sized 3^{rd} phase particles is of a speed avoiding precipitation of intermetallic compounds of CaₓMg_{y}Zn_{z} and recrystallization.

Further objects, features and advantages of this invention will become readily apparent to persons skilled in the art after review of the following description, including the claim, with reference to the drawings that are appended to and form a part of this specification.

### BRIEF DESCRIPTION OF THE FIGURES

FIG. 1 illustrates an atom probe reconstruction of BioMg 250 showing Ca and Zn atoms in clusters (scale is in nanometers).

FIG. 2A illustrates an electron micrograph (STEM) images of a GP zone in BioMg 250 in a bright-field image in which the bright atoms are Zn and Ca.

FIG. 2B illustrates an electron micrograph (STEM) images of a GP zone in BioMg 250 in a dark-field image in which the bright atoms are Zn and Ca.

FIG. 3. Diagrammatically illustrates an array of Zn and Ca atoms in a GP zone on a basal plane of the Mg matrix of BioMg 250.

FIG. 4 shows an atom probe analysis on a nanometer scale showing grain boundary films of CaₓMg_{y}Zn_{z} intermetallics and an adjacent zone denuded of clusters of Ca and Zn.

FIG. 5 illustrates an atom probe electron micrograph of cold worked/segregation treated BioMg 250, revealing Ca and Zn atom segregation to dislocations.

FIG. 6 illustrates an electron micrograph (HR-TEM) showing decorated dislocation domains in BioMg 250.

FIG. 7 is a bright-field electron micrograph image showing decorated dislocation domains in BioMg 250.

FIG. 8 illustrates spherical alpha Mn particles of 50 to 120 nm diameter for grain refinement.

FIG. 9 is a time/temperature/deformation diagram for BioMg 250 Grade 2, incorporating the principles of the present invention.

### DETAILED DESCRIPTION

In developing a new combination of alloying elements to fortify the mechanical properties of the Mg base, the search was narrowed first to elements that are established nutrients to the body, and then Quantum Mechanics First Principles were used to select and optimize ternary additions of odd-sized elements, both positive and negative sizes to the Mg atom and to each other. At the same time, microalloying principles and transition microstructures were practiced in order to capture synergisms amongst the alloying elements at low levels, while avoiding the detrimental effects on corrosion and ductility of equilibrium phases that are introduced by excessive alloying and faulty processing.

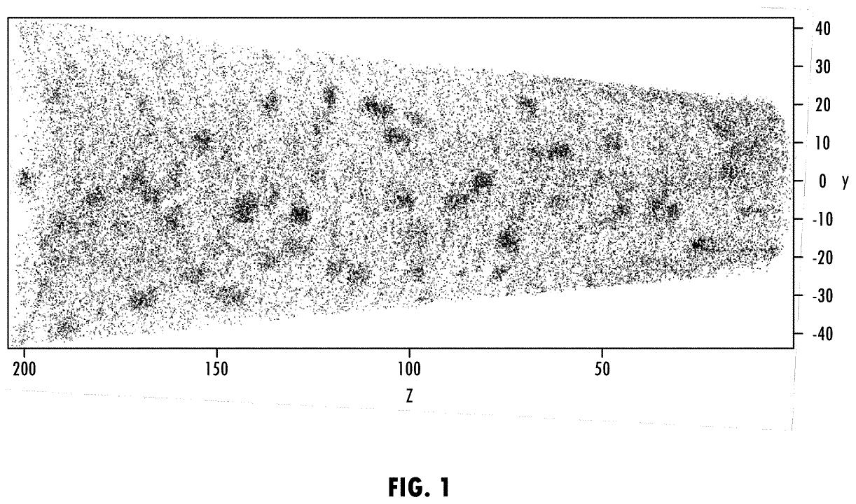

Based on the +/− atomic misfits and strengthening potencies seen below in Table I, the selected ternary alloying elements were zinc (Zn), manganese (Mn) and calcium (Ca). Zn, Mn and Ca achieve significant +/− oddness to Mg, respectively at −17, −14 and +23% in size, with a 32% oddness between Ca and Zn. Changing the equilibrium volume in opposite directions from the Mg matrix fosters co-segregation of Ca (which is positive to Mg) with Zn (which is negative to Mg). The mixing enthalpy between Ca and Zn is negatively large at −22 kJ/mol, an order of magnitude larger than Mg—Ca. Thus, there is a strong attractive interaction between Ca and Zn in the Mg matrix, which results in the forming clusters as seen in FIG. 1. Also, the pairing of large and small solute elements lowers misfit of a new phase with the matrix, enabling easier nucleation of transition nanostructures. Thus, the alloy is strengthened.

TABLE I
Alloying Elements in BioMg 250 and Misfits from Mg atom

| Alloying | % Size | | Equilibrium | |
|---|---|---|---|---|
| Element in | Misfit | Electronegativity | Volume | Strengthening |
| Mg | from Mg | Misfit from Mg | vs. Mg, | Potency, |
| Base | Atom | Atom | A³/atom | MPa/Atomic % |
| Zn | −17 | +0.5 | − | 33 |
| Ca | +23 | −0.2 | + | 84 |
| Mn | −14 | +0.4 | − | 121 |

Mg has poor ductility and formability, but ternary microalloying of Mg with both Ca and Zn improves both properties, more so than binary additions of Ca or Zn. This is related to reduced basal texture and enhanced non-basal slip.

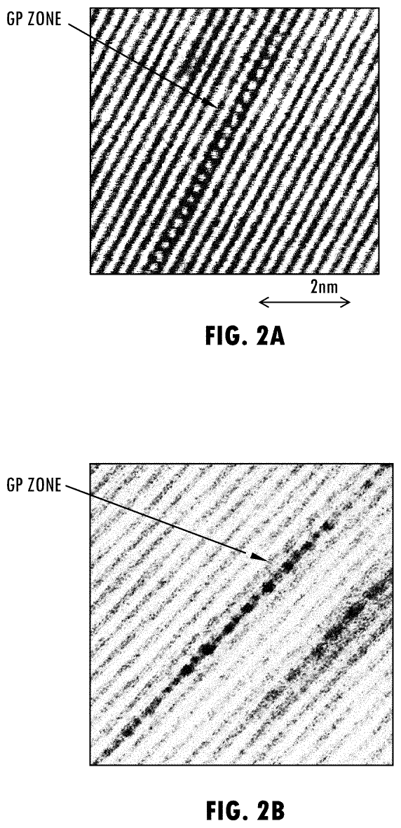

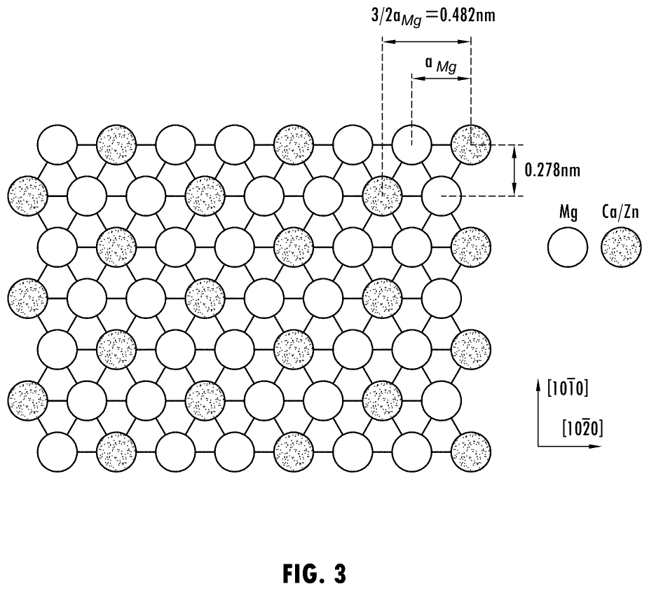

Indeed, strengthening and ductilizing is the case wherein clusters (as seen in FIG. 1) or short range ordered zones (known as Guinier-Preston (GP) zones) form on the basal {0001} planes of Mg. These GP zones are one atomic layer thick (<0.5 nm) and about 15 nm in diameter (see FIGS. 2A and 2B). An array of Zn and Ca atoms in these ordered zones is diagrammatically illustrated in FIG. 3. The Zn to Ca ratio is 2:1 and the interzone distance is 10 nm normal to the basal planes. The GP zone population is about 10²² to 10²³/m³. These clusters and zones resist dislocation deformation on the basal planes. The end result is referred to herein as BioMg 250 Grade 1, which is recrystallized after working by annealing for 1 hour at 400° C. and then aging for 2 hours at 200° C. to form clusters and/or precipitate GP zones, with the following range of properties:
a. yield strength of 130 to 149 MPa;
b. tensile strength of 255 to 168 MPa;
c. elongation of 22 to 28%; and
d. grain sizes of 13-15 μm.

Further to their benefit to mechanical properties of Mg, microalloying with Zn, Mn and Ca benefits the corrosion resistance of Mg. However, excessive additions of each element, or excessive combinations, are detrimental.

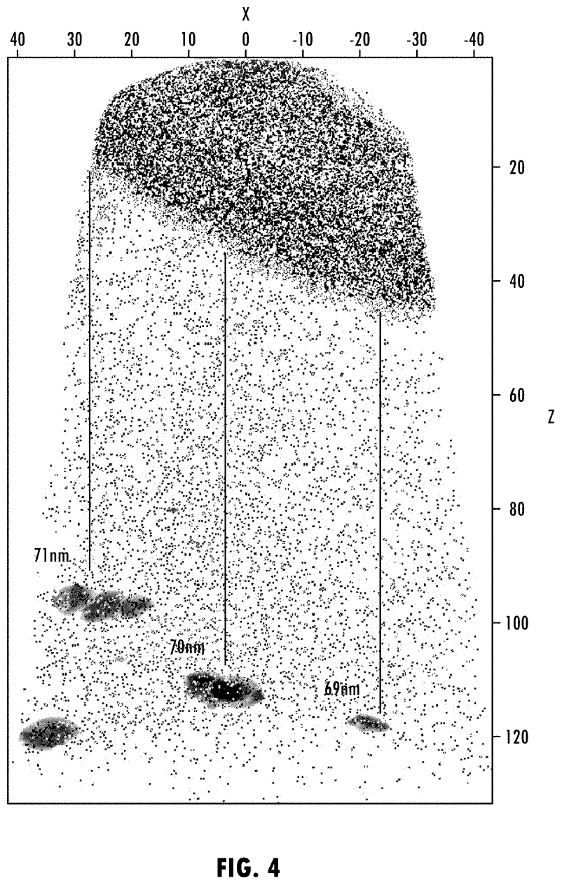

If one attempts to increase the strength of annealed/recrystallized BioMg 250 Grade 1 by extending the aging time to 20-50 hours, grain boundary films of CaₓMg_{y}Zn_{z} intermetallics develop, as seen in FIG. 4. Their composition is about 58% Mg, 28% Zn and 14% Ca (weight %), thus sapping the matrix of cluster forming Ca and Zn in a 70 nm wide denuded volume adjacent to the grain boundary (again, see FIG. 4). This damages ductility, formability and corrosion resistance.

The strength level of BioMg 250 Grade 1 is insufficient for various applications, such as self-tapping screws, rigid plates and devices that compete with titanium (Ti) and stainless steel (SS) implants. Accordingly, innovation was required in order to boost the strength level of the base BioMg 250 composition.

As discussed herein, novel thermomechanical processing routes have been discovered that utilize deformation induced decorated dislocation domain (DDD) nanostructures to boost yield strength above 220 MPa, while controlling the degradation rate in body fluids. As further discussed herein, the generation of dislocations and segregation/decoration can be simultaneous under specific strain rates and temperatures of hot deformation and post thermal treatment. Alternatively, dislocations can be generated by specific cold deformation followed by thermally activated segregation/decoration. Both of the above are herein referred to as “deformation and segregation treatments.”

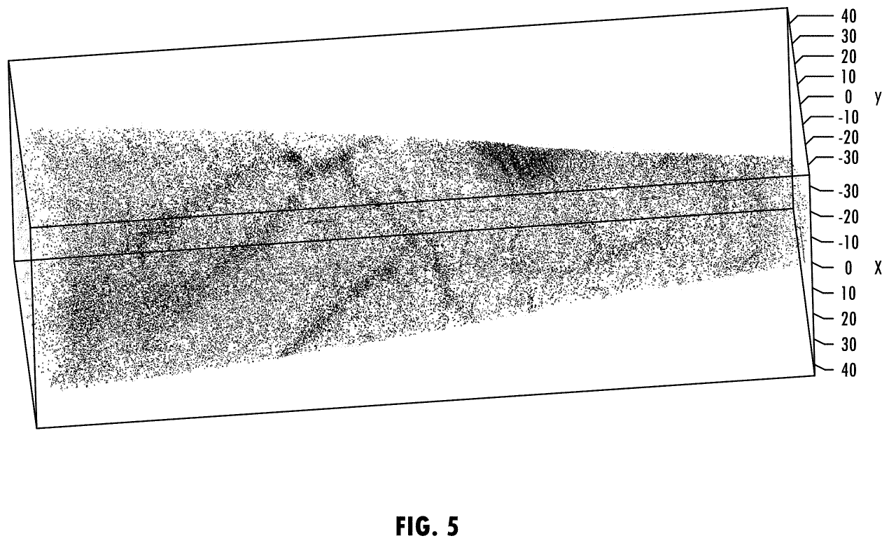

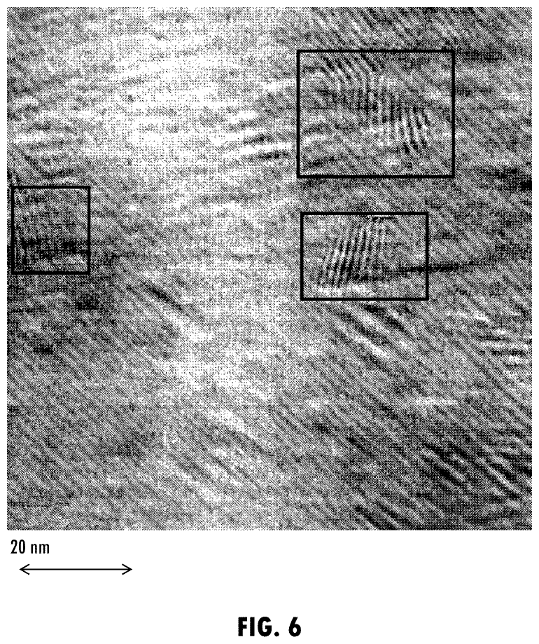

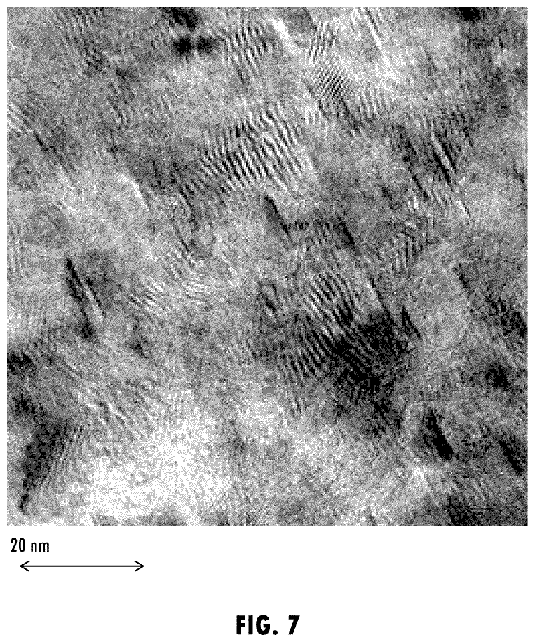

Introduced in the resultant post processing Mg alloy material are nanometer-sized DDD's comprising a) dense line and screw dislocations and stacking faults, b) dislocation arrays at the domain boundaries, c) multi-atom clusters on those dislocations (see FIG. 5) and d) coherency strains in layered planar arrays. As seen in FIGS. 6 and 7, the DDD's, about 20 nm in size, are disoriented from the mother grains. The above is achieved while at the same time avoiding significant recrystallization, grain boundary intermetallics and grain boundary denuded zones (see FIG. 4). These DDD nano-mechanisms add more strength than thin GP zones. With this microalloying approach, the Ca and Zn are more efficiently dedicated to useful nano-strengthening, rather than to wasteful and deleterious gross intermetallic forms at grain boundaries. Texture (unfavorable crystallinity orientation) can be decreased. The fine structure and low texture also favor low corrosion. Ca and Zn lower the stacking fault energy of Mg. The novel DDD nanostructure results in activation of non-basal slip, i.e. pyramidal and/or prismatic slip, increasing grain boundary cohesive energy, 2γᵢₙₜ, to the benefit of increased ductility, formability and toughness.

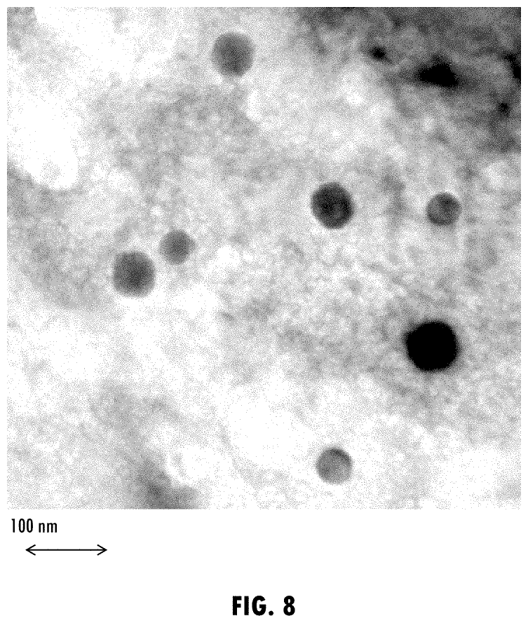

In addition to its role in solid solution hardening, Mn additions insert spherical nm size alpha Mn particles that refine the grain size and amplify the Hall-Petch strengthening (see FIG. 8).

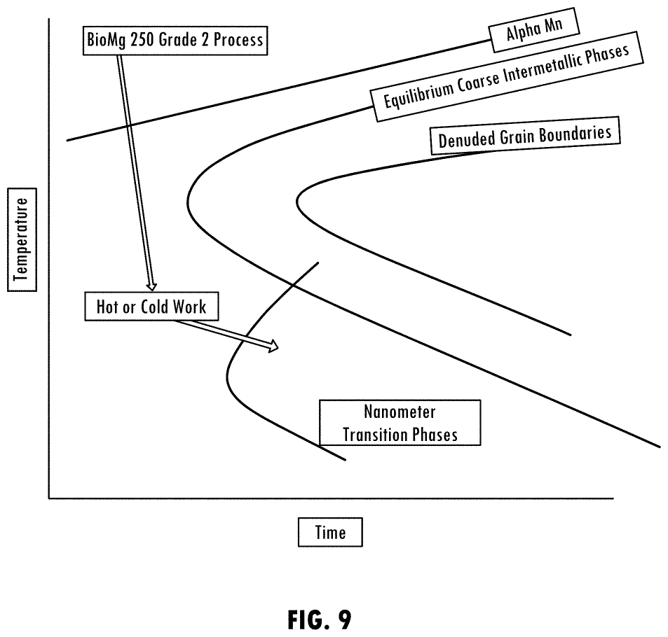

As shown in FIG. 9, the alpha Mn reaction is activated before the deformation steps. This time/temperature/deformation processing route is structured to avoid equilibrium phases, while promoting transition phases. According to FIG. 9, processing is practiced as follows:
a.) the alloy is cooled through the alpha Mn precipitation temperature to precipitate that phase in a fine array of nanometer size;
b.) the alloy is cooled fast enough to avoid the precipitation of equilibrium CaₓMg_{y}Zn_{z} intermetallics, especially at the grain boundaries; likewise, fast cooling avoids the denuded grain boundaries adjacent to the grain boundary intermetallics;
c.) hot working is applied to generate dislocations and to afford rearrangement into arrays as they are decorated by segregation of diffusing odd-sized atoms, while avoiding grain growth;
d.) alternately, cold working of the Mg matrix is applied to generate the dislocations; and
e.) finally, the alloy is held at low temperatures for short times so as to transform to nanometer transition phases and microstructures (these microstructures are decorated by segregating large and small atoms in the form of clusters, GP zones and dislocation domains).

The formed transition phases resist dislocation movement; thus increasing strength as clouds on individual dislocations of line, screw and partial types. But also, as seen above, the 20 nanometer-sized decorated dislocation domains are disoriented from the mother grains, as enabled by dislocation walls at their boundary. This disorientation resists easy dislocation slip on the basal plane of Mg, activating additional slip on pyramidal and/or prismatic planes. This results in ductility and formability being improved, while at the same time increasing strength.

### EXAMPLES

The following examples utilize BioMg 250, whose composition by weight % is Mg Base, 1.33 Zn, 0.39 Ca and 0.46 Mn.

### Example 1—Strengthening by Cold Deformation

Cold working in accordance herewith is very effective in boosting the strength of BioMg 250. This step is introduced prior to the segregation treatment noted above. This room temperature deformation can be applied by stretching, drawing, spinning and/or rolling. Results achieved by cold stretching sheet material with a grain size of 16 microns are reported in Table II. Strength (YS, UTS) increased and elongation (El) decreased as the segregation treatment factor (F) (defined below) increased. In addition, prior thermal treatment to attain finer grain size enhanced the combination of strength and elongation (see Table III). The highest elongation was seen with a pre-cold work Thermal Treatment Factor (TTF) of 10.72, wherein:

TTF=Temperature (° K/1000)×{18+log time (hours)} (1)

TABLE II
BioMg 250 Sheet Cold Worked by Stretching + Segregation Treatment
(Grain Size = 16 microns)

| Segregation Treatment | | | |
|---|---|---|---|
| Factor, F | YS, MPa | UTS, MPa | El, % |
| 1.77 × 10⁵ | 233 | 268 | 21 |
| 1.88 × 10⁵ | 243 | 276 | 20 |
| 3.2 × 10⁵ | 273 | 280 | 13 |
| 3.4 × 10⁵ | 272 | 290 | 12 |

TABLE III
Effect of Prior Thermal Treatment on BioMg 250 Sheet
Cold Worked by Stretching + Segregation Treatment Factor
of 1.7 × 10⁵

| Thermal | | | | |
|---|---|---|---|---|
| Treatment | | | | |
| Factor, | | | | Grain |
| TTF | YS, MPa | UTS, MPa | EL., % | Size, μm |
| 9.86 | 299, 300, 308 | 304, 305, 308 | 11, 7, 8 | 5 |
| 10.72 | 293, 298, 304 | 297, 298, 306 | 19, 14, 15 | 8 |
| 12.11 | 289, 299 | 291, 300 | 11, 8 | 16 |

The results with cold bar drawing are listed Table IV. The effect of % cold work is listed in Table V, wherein cold work increases strength and decreases work hardening. The segregation treatment is quantified by a factor, F, the product of its key variables, namely: a) % of prior deformation, b) time of segregation treatment in minutes and c) temperature of segregation treatment in ° K. For example, treatment may include a 20% deformation, 30 minutes heat treatment at 500° K, resulting in a segregation treatment F of 3.0×10⁵.

TABLE IV
Properties of Cold Drawn BioMg 250 Bar + Segregation Treatment

| Segregation Treatment | | UTS, | | | |
|---|---|---|---|---|---|
| Factor, F | YS, MPa | MPa | El, % | RA, % | Hv |
| 3.2 × 10⁵ | 314, 318 | 318, 325 | 12, 13 | 22, 26 | 69 |
| 1.7 × 10⁵ | 321, 325 | 322, 325 | 11, 11 | 31, 32 | 66 |

TABLE V
Effect of % Cold Work by Stretching BioMg 250 sheet -
on Strength, Work Hardening (UTS/YS) and Lankford No.

| Segregation | | | | | Work | |
|---|---|---|---|---|---|---|
| Treatment | % Cold | YS, | UTS, | El., | Hardening | Lankford |
| Factor, F | Work | MPa | MPa | % | UTS/YS | Number |
| Grade 1 | 0 | 142 | 246 | 28 | 1.73 | 0.88 |
| 5.4 × 10⁴ | 2 | 156 | 248 | 26 | 1.59 | 0.68 |
| 1.1 × 10⁵ | 4 | 187 | 262 | 18 | 1.32 | 0.72 |
| 1.8 × 10⁵ | 7 | 237 | 276 | 18 | 1.16 | 0.76 |
| 3.3 × 10⁵ | 12.5 | 272 | 285 | 13 | 1.05 | 0.82 |

The effect of higher % cold work is presented in Table VI, which shows that hardness increased with an increase in % cold work.

TABLE VI
Effect of % Cold Work on Segregated Hardness
of BioMg 250

| Segregation | % Cold | Hardness, |
|---|---|---|
| Treatment Factor, F | Work | Hv |
| 1.6 to 7.1 × 10⁵ | 12 | 63-69 |
| 3.0 to 13.6 × 10⁵ | 24 | 70-76 |

Further practice of cold work was applied to wire of BioMg 250, as seen in Table VII. Fine wire of 100 μm diameter was cold drawn to attain yield strength of 400 MPa, with 3-4% elongation.

TABLE VII
Mechanical Properties of BioMg 250 Wire

| Diameter, mm | YS, MPa | UTS, MPa | Elongation, % | Bendability |
|---|---|---|---|---|
| 1.1 | 400 | 400 | 3 | 2.4 mm radius |
| 0.3 | 400 | 400 | 3 | <1.2 radius |
| 0.1 (100 microns) | 400 | 400 | 4 | Tight knot |

As to the mechanism of cold work strengthening, dislocation introduction and decoration with Ca and Zn atoms was confirmed. Atom probe electron microscopy revealed that dislocations had been introduced by cold work and that Ca and Zn atoms had segregated to those dislocations, as seen in FIG. 5, to retard dislocation movement, thus strengthening the alloy. The segregation could occur during the adiabatic heating resulting from deformation.

As previously noted, the fine microstructure and low texture favor improved corrosion resistance. The corrosion resistance of BioMg 250 Grade 2 was improved by the cold work/segregation interjection in the processing (see Table VIII), as tested in Synthetic Body Fluid and measured by H₂ evolution and by potentiodynamic tests.

TABLE VIII
Corrosion Tests Results on Grade 1 and 2 BioMg 250 in SBF

| | | | Potentiodynamic | |
|---|---|---|---|---|
| | H₂ Rate | H₂ Rate | Corrosion | Potentiodynamic |
| | (mL · | (mm · | Current I | Corrosion Rate |
| Sample | cm⁻² · d⁻¹) | year⁻¹) | (μA · cm⁻²) | (mm · year⁻¹) |
| Grade 1 | 0.042 | 2.31 | 49.3 | 1.13 |
| Grade 2 | 0.019 | 1.03 | 40.7 | 0.93 |

Avoidance of grain boundary intermetallics and resultant denuded grain boundaries, seen in FIG. 4, lowers corrosion rates since such arrays set up galvanic corrosion between the intermetallic phase and the depleted zone.

### Example 2—Hot Deformation Plus Segregation

In addition cold deformation, hot deformation plus segregation treatment is effective in increasing strength. Dislocations are introduced and decorated at controlled strain rates and temperatures dependent on rate of dislocation movements and diffusion rates of the segregating elements. Residual dislocations from hot working were retained in Grade 2 material by avoiding recrystallization that occurs during the 1 hour anneal at 400° C. used on Grade 1. Dislocation contents on the order of 10¹⁴ to 10¹⁵ m⁻³ were introduced. These dislocations were decorated by Ca—Zn clusters. In addition, grain growth was restricted to retain grain sizes of 2-3 μm, much finer than the 13-18 μm sizes of Grade 1. Thus, the Hall-Petch strengthening mechanism was utilized wherein strength is inversely proportional to the square root of the grain diameter. The results from such residual hot deformation are revealed in Table IX. Yield strengths above 267 MPa with elongations above 9% were gained, as enabled by decorated dislocations and grain sizes of 3 μm or less. Greater hot reduction in the last rolling pass (e.g. 50%) introduced more dislocations and led to higher yield strength.

TABLE IX
BioMg 250 Hot Rolled Sheet with Segregation Treatment

| Segregation | | | | | Grain |
|---|---|---|---|---|---|
| Treatment | | UTS, | | Hardness, | Size, |
| Factor, F | YS, MPa | MPa | El, % | Hv | μm |
| 1.15 × 10⁵ | 273, 276, | 296, 298, | 17, 17, | 74, 75 | 2 to 3 |
| | 267, 285 | 299, 302 | 17, 19 | | |
| 2.86 × 10⁵ | 272, 279, | 296, 301, | 13, 18, | 76, 76 | 2 to 3 |
| | 278, 291 | 300, 304 | 14, 17 | | |
| 4.30 × 10⁵ | 295, 301, | 300, 304, | 11, 15, | 73, 76 | 2 to 3 |
| | 297, 312 | 313, 322 | 9, 11 | | |

A two-step segregation treatment, introducing dislocations during hot deformation and then segregating at a lower temperature, was also seen to be effective in reaching a yield strength of 291 MPa with good elongation (see Table X).

TABLE X
Two-Step Process on BioMg 250 Sheet

| Segregation Treatment Factor | YS, MPa | UTS, MPa | El., % |
|---|---|---|---|
| Hot Deformation-3.8 × 10⁵ + | 291 | 307 | 14 |
| Segregation-1.7 × 10⁵ | | | |

### Example 3—Low Texture and Good Formability and Ductility of BioMg 250 Grade 2

The large Ca atoms and small Zn atoms co-segregate to grain boundaries in a strong interaction. The large Ca atoms segregate to the extension region of dislocations in the grain boundaries, while the small Zn atoms segregate to the compression region—both minimizing the elastic strains of the dislocations in the grain boundaries. By this mechanism, grain growth of highly oriented <1120> grains is inhibited; thus randomizing the growth of grains with other orientations. Also contributing is the presence of second phase Mn particles during thermomechanical processing. Rather than domination of basal slip, deformation is shared on prism and pyramid planes. This makes for superior formability compared to conventional Mg alloys, which exhibit higher textures of the multiple of a random distribution, MRD, of about 10 in examination of solid test specimens by electron diffraction.

### Example 4—Other Odd-Sized Elements

Ca, Zn and Mn are preferred additions to Mg for microalloying of bioabsorbable Mg alloys. However, the present concept opens the door for structural Mg alloys with alternate combinations of big and small atoms that will segregate on dislocations. In a broad scope of the invention, alternate candidates to supplement or replace Ca and Zn are identified in Table XI.

TABLE XI
Atomic Radius and Electronegativity of Mg and Alloying
Elements (Reference-Periodic Table-Bar Charts, Inc.)

| | Atomic Radius | |
|---|---|---|
| Element | (Angstroms) | Electronegativity |
| Base Mg | 160 | 1.2 |
| Large Atoms | | |
| Ca | 197 | 1.0 |
| Sr | 215 | 1.0 |
| Y | 178 | 1.1 |
| Rare Earths | 173-199 | 1.0-1.1 |
| Ba | 217 | 1.0 |
| Na | 186 | 1.0 |
| K | 227 | 0.9 |
| Small Atoms | | |
| Zn | 133 | 1.7 |
| Mn | 137 | 1.6 |
| Sn | 141 | 1.7 |
| V | 131 | 1.8 |
| Cr | 125 | 1.6 |
| Al | 143 | 1.5 |
| Ag | 145 | 1.4 |
| Si | 118 | 1.7 |
| P | 110 | 2.1 |
| B | 80 | 2.0 |

The above description is meant to be illustrative of some preferred implementations incorporating the principles of the present invention. One skilled in the art will really appreciate that the invention is susceptible to modification, variation and change without departing from the true spirit and fair scope of the invention, as defined in the claims that follow. The terminology used herein is therefore intended to be understood in the nature of words of description and not of limitation.

We claim:

1. A strengthened Mg based alloy comprising: Mg as a base element and at least two microalloying elements; a microstructure having at least one of dislocations, stacking faults, coherency strains, grain boundaries and dislocation domains decorated by segregation of microalloying elements; one of the microalloying elements is a large atom element having an atomic size larger than the atomic size of a Mg atom; and another of the microalloying elements is a small atom element having an atomic size smaller than the atomic size of the Mg atom.

2. The Mg based alloy according to claim 1, further comprising an additional microalloying element providing separate nanometer-sized 3^{rd} phase particles that inhibit grain growth.

3. The Mg based alloy according to claim 2, wherein the additional alloying element is Mn and the separate nanometer-sized 3^{rd} phase particles are alpha Mn particles.

4. The Mg based alloy according to claim 2, wherein the 3^{rd} phase particles are in the range of 10 to 200 nanometers.

5. The Mg based alloy according to claim 2, wherein the 3^{rd} phase particles have a solvus at a higher temperature than equilibrium intermetallic compounds of the microstructure.

6. The Mg based alloy according to claim 1, wherein the microstructure includes an absence of continuous films of grain boundary intermetallic compounds.

7. The Mg based alloy according to claim 1, wherein the microstructure includes an absence of denuded grain boundaries.

8. The Mg based alloy according to claim 1, wherein atoms of the large atom element have atomic radii of 173 Angstroms or more and atoms of the small atom element have atomic radii of 145 Angstroms or less.

9. The Mg based alloy according to claim 1, wherein the atoms of the large atom element have an electronegativity of 1.1 or less and atoms of the small atoms element have an electro negativity of 1.4 or more.

10. The Mg based alloy according to claim 1, wherein the large atom element is Ca and the small atom element is at least one of Zn and Mn.

11. The Mg based alloy according to claim 10, where the microalloying elements essentially consist of Zn, Ca and Mn in the ranges (weight %) of 0.7 to 1.8 Zn, 0.2 to 0.7 Ca and 0.2 to 0.7 Mn.

12. The Mg based alloy according to claim 10, where the microalloying elements essentially consist of Zn, Ca and Mn in the ranges (weight %) of 0.5 to 2.0 Zn, 0.2 to 1.0 Ca and 0.2 to 1.0 Mn.

13. The Mg based alloy according to claim 1, where the base and microalloying elements are human body nutrients and are osteoconductive

14. The Mg based alloy according to claim 1, where the Mg alloy is provided in the form of a bioabsorbable, human or animal body implant.

15. The Mg based alloy according to claim 14, wherein the Mg alloy is provided in the form of a structural reinforcement device.

16. The Mg alloy of claim 1, wherein dislocation and partial dislocation content is retained at greater than 10¹³/m³.

17. The Mg alloy of claim 1, wherein yield strength of the Mg alloy is greater than 220 MPa.

18. The Mg alloy of claim 1, wherein texture of the Mg alloy is below an MRD value of 5 and formability is enhanced by an r value of less than 2.

19. The Mg alloy of claim 1, wherein the Mg allow includes at least one of intragranular GP zones of less than 100 nm in size and intragranular intermetallic particles of less than 200 nm in size.

20. The Mg alloy of claim 1, wherein the Mg alloy further includes segregated planar layers of alloying elements resulting in coherency strains supplementing the strength of the Mg alloy.

21. A method of processing a bioerodible magnesium alloy containing at least 95 weight percent magnesium in combination with microalloying elements for forming an endoprosthesis device, the method comprising:
forming one of an ingot or billet comprised of the magnesium alloy,
strengthening the magnesium alloy by applying a deformation and segregation treatment, the treatment forming at one of dislocations, grain boundaries, stacking faults, clusters, GP zones and/or dislocation domains that are decorated by segregation of microalloying elements,
at least one element of the microalloying elements is a large atom microalloying element having atoms with an atomic size larger than a magnesium atom,
at least another element of the microalloying elements is a small atom microalloying element having atoms with an atomic size smaller than the magnesium atom, and
forming in the microstructure separate grain growth inhibiting nanometer-sized 3^{rd} phase particles of an additional microalloying element.

22. The method of claim 21, further comprising the step of forming one of a rod, wire, hollow tube, mesh, scaffold and sheet from the ingot or billet.

23. The method of claim 21, further comprising the step of forming the bioerodible magnesium alloy into an endoprosthesis implant in the form of at one of a screw, plate, wire, mesh, scaffold and stent.

24. The method of claim 21, wherein the large atom microalloying element is one or more of Ca, Sr, Ba, Na, K, RE and Y and the small atom microalloying element is of one or more of Zn, Mn, Sn, V, Cr, P, B, Si, Ag and Al.

25. The method of claim 21, where deformation is by at least one of cold drawing, cold stamping, cold stretching, cold swaging, cold spinning or cold rolling.

26. The method of claim 21, where the deformation is by at least one of hot extrusion, hot rolling, hot pressing, hot swaging, hot spinning or hot forging wherein the rolls or dies are heated to 150 to 400° C. and the deformation is greater than 0.3.

27. The method of claim 21, wherein the forming step includes forming decorated dislocations of grain sizes less than 5 μm and decorated dislocation domains of less than 50 nanometers.

28. The method of claim 21, wherein the product of percent deformation×time (minutes)×temperature (° K) in the segregation treatment is between 5×10⁴ and 5.6×10⁵ for hot deformation and between 8×10⁴ and 21×10⁵ for cold deformation.

29. The method of claim 23, wherein the deformation and segregation treatment includes cold working producing adiabatic heating resulting in segregation.

30. The method of claim 21, wherein the additional microalloying element is Mn and the process of deformation and segregation treatment in the presence of the nanometer-sized 3^{rd} phase particles is of a speed avoiding precipitation of intermetallic compounds of CaₓMg_{y}Zn_{z} and recrystallization.
